(19) United States
(12) Patent Application Publication
(10) Pub. No.: US 2022/0046928 A1
(43) Pub. Date: Feb. 17, 2022

(54) USE OF GUAR DERIVATIVES IN BIOFUNGICIDE COMPOSITIONS

(71) Applicant: RHODIA OPERATIONS, Aubervilliers (FR)

(72) Inventors: Jean-Christophe CASTAING, Sèvres (FR); Florence LAMBERT, Paris (FR); Mirjana GELO-PUJIC, Sérézin-du-Rhône (FR)

(21) Appl. No.: 17/298,290

(22) PCT Filed: Nov. 29, 2019
(86) PCT No.: PCT/EP2019/083155

§ 371 Date: May 28, 2021

Related U.S. Application Data
(60) Provisional application No. 62/772,828, filed on Nov. 29, 2018.

(51) Int. Cl. A01N 63/23; A01N 63/27; A01N 63/34; A01N 63/36; A01N 63/38; A01N 43/16; A01N 25/02

(52) U.S. Cl. CPC A01N 63/23; A01N 63/27; A01N 63/34; A01N 25/02; A01N 63/38; A01N 43/16; A01N 63/36

(57) ABSTRACT

The present invention relates to a fungicide composition comprising a biofungicide and a guar derivative containing at least one hydroxyalkyl group.

This application claims priority to USPA No. 62/772828 filed on Nov. 29, 2018, the whole content of this application being incorporated herein by reference for all purposes.

The present invention concerns the use of guar derivatives in bio fungicide compositions, especially the use of guar derivatives for the growth of microorganisms, in particular of suppressive microorganisms.

Diseases caused by fungal species are considered among the most widespread and damaging of plants worldwide. Presently, control of plant fungal diseases is largely dependent upon the application of certain chemicals. Although some of these chemicals are known to have negative environmental and human health problems, nevertheless such chemical agents continue to be in wide use due to their strong activity against important fungal diseases, and limited availability of environmentally safer and effective alternatives.

Generally, biological control of diseases commonly infecting plants in the root zone (rhizosphere) and the leaf zone (phylloplane) are preferred over more traditional synthetic chemical control methodologies. Such biocontrol agents usually cause little or no injury to the plant host or the environment, and some may even favor normal plant development. However, most such biocontrol organisms are typically very limited either in the scope of their effectiveness against fungal diseases, or in their ability to survive and maintain the activity under formulation conditions, storage conditions, application under practical field conditions and during treatment applications.

Attempts have been made to control plant fungal diseases by using certain microorganisms.

Microorganisms used in agriculture are generally bacteria, yeasts, molds, mycorrhizae.

There is thus a need to find means to maintain or even improve the activity and efficiency of target microorganisms.

There is also a need to find means to specifically maintain or improve the growth of a target microorganism in a given medium.

More generally there is a need to provide formulations useful to selectively improve the growth of target microorganism in a given medium.

There is also a need to provide formulations useful to selectively stimulate a target microorganism in a given medium.

The inventors have found that a specific guar derivative was useful to address these needs.

Therefore, the present invention relates to fungicide composition comprising a bio-fungicide and a guar derivative containing at least one hydroxyalkyl group.

According to the invention, the guar derivative of the invention may increase the growth rate of the bio-fungicide.

The growth rate of microorganisms, in particular of suppressive microorganism, may be measured by the following method:

Microorganisms are incubated in a culture media in presence of guar. Sampling is performed at different times in order to determine the number of colony forming unit (CFU) using the spread-plating method. With this methodology, the evolution of the number of bacterial cells (expressed as CFU) as a function of time is obtained. The microorganism growth follows an exponential law: Nₜ=N₀e^{(μt)} with μ the growth rate of microorganisms. The value of the growth rate of microorganisms μ is obtained by fitting the experimental data in logarithmic scale, it corresponds to the slope of the evolution of ln(Nₜ) as a function of time (linear plot: ln(Nₜ)=ln(N₀)+μt).

According to an embodiment, the present invention relates to method for maintaining or increasing the growth rate of microorganisms, in particular of suppressive microorganism, comprising a step of contacting said microorganism with a guar derivative of the invention.

The present invention is based on the use of a guar derivative of the invention which enables to maintain and keep constant over the time the bio fungicide effect of microorganisms, in particular of suppressive microorganism, and in other words to maintain the growth rate of microorganisms, and in particular to maintain the bacterial growth rate.

Advantageously, the use of said guar derivative enables to increase the biofungicidal activity of microorganisms, in particular of suppressive microorganism, in other words the bio fungicidal activity of a microorganism is significantly or substantially greater when applied in association or combination with a guar derivative of the invention than that obtained without the use of said guar derivative.

Preferably, according to the invention, when using the guar derivative as defined above, the growth rate of microorganisms is increased of at least 5%, preferably of at least 10%, in comparison to the growth rate of microorganisms when no guar derivative is used.

The present invention also relates to use of a microorganism, in particular of a suppressive microorganism, and of a guar derivative containing at least one hydroxyalkyl group, as bio fungicide.

The present invention also relates to a kit comprising at least one microorganism, in particular a suppressive microorganism, and at least one guar derivative containing at least one hydroxyalkyl group, as well as to the use of said kit as biofungicide.

According to an embodiment, the present invention relates to a method for controlling fungal organism comprising applying a fungicide composition as described previously.

The present invention also relates to a method of controling or preventing infection of a plant by phytopathogenic fungi, comprising the step of applying to said plant a fungicide composition as described previously.

According to an embodiment, the guar derivative as mentioned above is used in a plant, on a seed or in the soil.

Throughout the description, including the claims, the term “comprising one” or “comprising a” should be understood as being synonymous with the term “comprising at least one”, unless otherwise specified, “between” and “from . . . to . . . ” should be understood as being inclusive of the limits.

As used herein, “weight percent,” “wt %,” “percent by weight,” “% by weight,” and variations thereof refer to the concentration of a substance as the weight of that substance divided by the total weight of the composition and multiplied by 100.

Should the disclosure of any patents, patent applications, and publications which are incorporated herein by reference conflict with the description of the present application to the extent that it may render a term unclear, the present description shall take precedence.

### Guars

Guars are polysaccharides composed of the sugars galactose and mannose. The backbone is a linear chain of β 1,4-linked mannose residues to which galactose residues are 1,6-linked at every second mannose in average, forming short side units.

The guar derivative of the invention contains at least one hydroxyalkyl group.

According to one of the invention embodiments, the hydroxyalkyl group is a C₁-C₆ hydroxyalkyl groups, for instance chosen from the group consisting of: a hydroxymethyl group, a hydroxyethyl group, a hydroxypropyl group and a hydroxybutyl group.

According to one of the invention embodiments, the hydroxyalkyl group is a hydroxypropyl group.

According to anyone of the invention embodiments, the degree of hydroxyalkylation (molar substitution or MS) of the guar derivative of the invention means the number of alkylene oxide molecules consumed by the number of free hydroxyl functions present on the polysaccharide.

According to anyone of the invention embodiments, the guar derivative of the invention has a degree of hydroxyalkylation (MS) higher than or equal to about 0.1, for instance higher than or equal to about 0.2.

According to anyone of the invention embodiments, the guar derivative of the invention has a degree of hydroxyalkylation (MS) lower than or equal to about 3.0, for instance lower than or equal to about 2.0.

According to anyone of the invention embodiments, the guar derivative of the invention has a degree of hydroxyalkylation (MS) comprised between about 0.1 and about 3.0, for instance between about 0.1 and about 2.0.

A guar derivative of the invention containing at least one hydroxyalkyl group may be prepared for example by reacting the corresponding alkene oxides (such as for example propylene oxides) with the guar so as to obtain a guar derivative which has been modified with hydroxyalkyl group (for example hydroxypropyl groups).

By the expression “average molecular weight” of the guar derivative of the invention, it is meant the weight average molecular mass of said guar derivative.

The average molecular weight of a guar derivative may be measured by SEC-MALS (Size Exclusion Chromatography with detection by Multi-Angle Light-Scattering detection). A value of 0.140 for dn/dc is used for the molecular weight measurements. A Wyatt MALS detector is calibrated using a 22.5 KDa polyethylene glycol standard. All calculations of the molecular weight distributions are performed using Wyatt's ASTRA software. The samples are prepared as 0.05% solutions in the mobile phase (100 mM Na2NO3, 200 ppm NaN3, 20 ppm pDADMAC) and filtered through 0.45 μm PVDF filters before analysis. The average molecular weights are expressed by weight.

According to anyone of the invention embodiments, the average molecular weight of the guar derivative of the invention is higher than about 100,000 g/mol, for instance higher than about 150,000 g/mol, for instance higher than about 200,000 g/mol.

According to anyone of the invention embodiments, the average molecular weight of the guar derivative of the invention is lower than about 3,000,000 g/mol.

According to one of the invention embodiments, the average molecular weight of the guar derivative is comprised between about 100,000 g/mol and about 3,000,000 g/mol, for instance between about 150,000 g/mol and about 3,000,000 g/mol, for instance between about 200,000 g/mol and 3,000,000 g/mol.

According to anyone of the invention embodiments, the guar derivative of the invention may further contain at least one cationic group.

As used herein, the term “cationic” covers not only positively charged groups, but also groups which may become positively charged depending on the pH.

A cationic guar derivative of the invention is a guar that has been chemically modified to provide said polysaccharide with a net permanent positive charge in a pH neutral aqueous medium. Those that are non permanently charged, e.g. guar derivatives that can be cationic below a given pH and neutral above that pH also fall within the scope of the present invention.

According to anyone of the invention embodiments, the terms “cationizing agents”, “cationic groups” and “cationic moieties” include ammoniums (which have a positive charge) but also primary, secondary and tertiary amines and their precursors (which can lead to positively charged compounds).

According to the invention, the guar derivative may be derivatized or modified so as to contain a cationic group.

According to one of the invention embodiments, the guar derivative of the invention may result from the reaction of any guar, with a cationizing agent.

Cationizing agents of the present invention are defined as compounds which, by reaction with the hydroxyl groups of the guar derivative can lead to a guar derivative comprising at least one cationic group according to the invention.

Cationizing agents of the present invention are defined as compounds which contain at least one cationic moiety. Cationizing agents comprise agents which can lead to cationic modified guars.

A group of suitable derivatizing reagents typically contain a reactive functional group, such as an epoxy group, a halide group, an ester group, an anhydride group or an ethylenically unsaturated group, and at least one cationic moiety or a precursor of such cationic moiety.

As used herein, the term “derivatizing agent” means an agent containing at least a cationic moiety which is grafted to a guar. The term “derivatizing agent” encompasses the terms “cationizing agent” and “grafting agent”.

In one embodiment of the invention, the cationic moieties may be linked to the reactive functional group of the derivatizing agent by a bivalent linking group, such as an alkylene or oxyalkylene group. Suitable cationic moieties include primary, secondary, or tertiary amino groups or quaternary ammonium, sulfonium, or phosphinium groups.

The derivatizing agent can comprise a cationic moiety, or a precursor of a cationic moiety, that contains a cationic nitrogen moiety, more typically, a quaternary ammonium moiety. Typical quaternary ammonium moieties are trialkylammonium moieties, such as trimethylammonium moieties, triethylammonium moieties, or tributylammonium moieties, aryldialkylammonium moieties, such as benzyldimethylammonium moieties, and ammonium moieties in which the nitrogen atom is a member of a ring structure, such as pyridinium moieties and imidazoline moieties, each in combination with a counterion, typically a chloride, bromide, or iodide counterion.

According to one of the invention embodiments, examples of cationizing agents, which lead to cationic guar derivatives of the invention are:
cationic epoxides, such as 2,3-epoxypropyltrimethylammonium chloride, 2,3-epoxypropyltrimethylammonium bromide, 2,3-epoxypropyltrimethylammonium iodide;
chlorohydrin-functional cationic nitrogen compounds, such as 3-halogeno-2-hydroxypropyl trimethylammonium chloride, for example 3-chloro-2-hydroxypropyl trimethylammonium chloride,
cationic ethylenically unsaturated monomers or their precursors, such as trimethylammoniumpropyl methacrylamide chloride salt, trimethylammoniumpropyl methacrylamide methylsulfate salt, diallyl dimethyl ammonium chloride, vinyl benzyl trimethylammonium chloride, dimethylaminopropyl methacrylamide (tertiary amine) precursors of cationic monomers, such as N-vinyl formamide, N-vinylacetamide (whose units can be hydrolyzed after polymerization or grafted onto vinyl amine units).

In one embodiment of the invention, the cationizing agents, which lead to cationic guar derivatives of the invention are cationic epoxides, such as 2,3-epoxypropyltrimethylammonium chloride, 2,3-epoxypropyltrimethylammonium bromide and 2,3-epoxypropyltrimethylammonium iodide.

According to the invention, the cationic groups may be introduced into a guar by reacting the guar starting material with a derivatizing agent which comprises a reactive functional group and at least one cationic moiety (or a precursor of cationic moiety).

According to the invention, the cationic groups present in the guar derivative are incorporated into the guar starting material by reaction of the hydroxyl groups of said guar with a cationizing agent.

Preferred cationic groups are chosen from the group consisting of: primary, secondary or tertiary amino groups, quaternary ammonium, sulfonium or phosphinium groups, and mixtures thereof. In a particular preferred embodiment, the cationic group is chosen from trialkylammonium groups, such as trimethylammonium groups, triethylammonium groups, tributylammonium groups, aryldialkylammonium groups, such as benzyldimethylammonium groups, and ammonium groups in which the nitrogen atom is a member of a ring structure, such as pyridinium groups and imidazoline groups, each in combination with a counterion, typically a chloride, bromide, or iodide counterion. Preferably, each cationic group contains at least one cationic charge.

The cationicity of the guar derivative can be expressed in terms of degree of substitution.

As used herein, the expression “cationic degree of substitution” (DScat) means the average number of moles of cationic groups per mole of sugar unit. The (DScat) may be measured by means of 1H-NMR (solvent : D2O).

Once the 1H NMR spectrum is obtained, the integration of the multiplet of peaks corresponding to the anomeric proton on all guar units, usually between 3.2-4.3 ppm, is normalized to unity. The peak of interest, the one corresponding to the methyl protons of the quaternary ammonium group on guar units, is centered around 1.8 ppm. This peak is integrated for 9 protons given that there are 3 methyl groups on the ammonium function. Therefore the calculation of the (DScationic) for the case of the cationizing agent 2,3-epoxypropyltrimethylammonium chloride is as follows:

$$DS = \frac{INTEGRAL\_N ( Me ) 3}{INTEGRAL\_anomeric \_proton} / 9$$

According to one of the invention embodiments, the guar derivative of the invention has a cationic degree of substitution (DScat) equals to zero.

According to another of the invention embodiments the guar derivative of the invention has a cationic degree of substitution (DScat) higher than or equal to about 0.02, for instance higher than or equal to about 0.05, for instance higher than or equal to about 0.08, for instance higher than or equal to about 0.09, for instance higher than or equal to about 0.10.

According to anyone of the invention embodiments, the guar derivative of the invention has a cationic degree of substitution (DScat) lower than or equal to about 3.0.

According to a specific embodiment, the guar derivative of the invention may be a hydroxypropyl guar hydroxypropyltrimonium chloride.

The degree of hydroxyalkylation (molar substitution or MS) of guar derivative containing at least one hydroxyalkyl group., that is the number of alkylene oxide molecules consumed by the number of free hydroxyl functions present on the guar, may be comprised between 0 and 3, preferably between 0 and 1.7. As example, a MS of 1 may represent one ethylene oxide unit per monosaccharide unit.

According to an embodiment, the Degree of Substitution (DS) of guar derivatives, that is the average number of hydroxyl groups substituted per sugar unit, is comprised between 0.005 and 3. DS may notably be determined by titration. The guar derivative of the present invention may have a DS of between 0.005 and 2. Preferably, the guar derivative of the present invention has a DS of between 0.005 and 1. More preferably, the guar derivative of the present invention has a DS of between 0.12 and 0.5.

The Charge Density (CD) of guar derivatives may be comprised between 0.01 and 4.9 meq/g, preferably between 0.4 and 2.1 meq/g. The charge density refers to the ratio of the number of positive charges per gram of polymer. For example, CD=1 meq/g means there are 0.001 charges per gram of polymer. The charge density multiplied by the polymer molecular weight determines the number of positively charged sites on a given polymer chain.

According to the present invention, the guar derivative may have an average molecular weight (Mw) of between about 2,000 Daltons and 90,000 Daltons, preferably, the guar derivative has an average molecular weight of between about 5,000 Daltons and 90,000 Daltons, more preferably, the guar derivative has an average molecular weight of between about 10,000 Daltons and 60,000 Daltons, still more preferably, the guar derivative has an average molecular weight of between about 10,000 Daltons and 50,000 Daltons.

The guar derivative according to the present invention may be prepared by depolymerizing cationically modified guars that have high molecular weight, so as to “split” the guar polymers to desired sizes. It is appreciated that the guar derivative of the present invention may also be prepared by depolymerisation of natural guars, followed by cationization reactions to provide the polymers with cationic functionality. Various depolymerisation methods are well known in the art and may be used for the present invention, such as treatment by using peroxo compound (e.g., hydrogen peroxide) and irradiation. Examples of such methods are disclosed in U.S. Pat. Nos. 4,547,571, 6,383,344 and 7,259,192.

The cationization of guars can be easily made by a skilled person using methods commonly known in the art. Various methods for providing guar gums with cationic functionality are known in the art, for example as disclosed in U.S. Pat. Pub. No. 2008/0112907. Various methods for cross-linking guars with and without cationic modification of the guars are also known, see for example U.S. Pat. Nos. 5,532,350 and 5,801,116. Alternatively, low molecular weight guars can be obtained by harvesting guar beans which are still at an early developmental stage such that the harvested guar beans contain low molecular weight natural guar gums. Then the guar gums may be subject to cationization to provide them with cationic functionality.

The guar derivatives as defined above may be used in a composition.

The composition containing the guar derivative may be a solid or a liquid composition. In the case wherein the composition is solid, the composition may be in the form of a powder, a particle, an agglomerate, a flake, a granule, a pellet, a tablet, a brick, a paste, a block such as a molded block, a unit dose, or another solid form known to those of skill in the art. Preferably, the solid composition is in the form of a powder or a granule.

In some aspects, the composition containing the guar is in the form of a granule. Granules containing the guar derivative may be prepared in a three-step procedure: wet granulation followed by drying and sieving. The wet granulation step notably involves introduction and mixing of guar derivative powders and a carrier, and optionally other ingredients, in granulation equipment (such as a mixing granulator). The mixing is conducted with spraying of water to the mixture. The wet granulation step will yield wet granules containing the guar derivatives. The weight ratio between the carrier and the guar derivative which are to be mixed may be between 20:1 to 1:1, preferably, between 20:1 to 10:1. The water content introduced may be comprised between 10 wt % to 50 wt % based on the total weight of the wet granules. The carrier may be silicon dioxide, amorphous silica, precipitated silica, hydrated amorphous silica, precipitated silica, hydrated amorphous synthetic calcium silicate, hydro fobized precipitated silica, silica gel, sodium aluminium silicate, clay, zeolite, bentonite, layered silicate, caolim, sodium carbonate, sodium bicarbonate, sodium sulfate, sodium tripolyphosphate, sodium chloride, sodium silicate (water glass), magnesium chloride, calcium chloride, ammonium chloride, magnesium sulfate, calcium carbonate, calcium oxide, and/or calcium sulphate, or a mixture thereof. Notably, the carrier is selected from calcium chloride and calcium carbonate. The drying step notably involves drying the wet granules by using hot air flow. This step can usually be conducted in a fluid bed equipped with an air inlet and an air outlet. The sieving step may be conducted by using a vibrating plate.

The granules may have a diameter of 0.1 to 6 mm. Generally, normal granules have a diameter of 2-6 mm and micro granules have a diameter of 0.1-2 mm. Preferably, micro granules having a diameter of 0.5-1.6 mm are used.

Alternatively, the granules containing the guar derivative may be prepared by using extrusion methods well known by a person skilled in the art. The extrusion methods are described in U.S. Pat. No. 6,146,570. For example, the guar derivative and the carrier, and optionally other ingredients, may be blended with heating. The weight ratio between the carrier and the guar derivative may be between 20:1 to 1:1. Then a binder may be melted and introduced into the mixture of the guar derivative and the carrier. Then, an extrusion step may be carried out with extruder temperature maintained between 55° C. and 65° C. The soft warm granules may be formed and may be subsequently cooled below solidification point of the molten binder (at room temperature for instance) in order to obtain solid granules.

In the case that the composition (seed treatment composition or composition for foliar application) is liquid, the liquid composition may be a suspension, a dispersion, a slurry, a solution in a liquid carrier selected from water, organic solvents oils or a mixture thereof. The liquid composition may be prepared by mixing the guar derivatives as described above with the liquid carrier, optionally with other components, by using conventional methods. Preferably, the liquid composition is in the form of an aqueous solution. The composition may comprise from 1 wt % to 60 wt % of the guar derivative based on the total weight of the composition. Preferably, the composition comprises from 5 wt % to 35 wt % of the guar derivative based on the total weight of the composition. In some aspects, the composition comprises from 20 wt % to 30 wt % of the guar derivative based on the total weight of the composition. When conducting seed treatment in industrial scale, it is preferred that the liquid composition used for the seed treatment contains high concentration of the guar derivative, so that less volume of the liquid composition is required to achieve the desired dosage for the treatment (i.e. the weight ratio of the guar derivative to the seeds being treated). Using small volume of the liquid composition can save costs and is less tedious. However, when the concentration of the guar derivative in the liquid composition increases, the fluidity of the liquid composition will significantly decrease. As a result, the liquid composition may become too “thick” to be effectively applied to the seed or the soil, and has poor ability to spread on the surface of the seed or in the soil as well. For example, an aqueous composition comprising 3 wt % of a high molecular weight guar derivative may already be very thick and thus have poor fluidity. One advantage of the present invention is that the guar derivative according to the present invention has relatively low molecular weight. In such case, the resulting liquid composition can maintain excellent fluidity even if the guar derivative is present at high concentrations, and therefore, such liquid composition can be conveniently used for treating the seeds or the soil. In one embodiment, the method of the present invention comprises a step in which the seed is coated with the composition as described above. Then the coated seed may be applied onto or in the soil, notably, in order to set in contact the coated seed with the ground.

Suitable coating techniques may be utilized to coat the seed or agglomeration of the seeds with the composition according to the present invention. Equipment that may be utilized for coating can include but are not limited to drum coaters, rotary coaters, tumbling drums, fluidized beds and spouted beds. It is appreciated that any suitable equipment or technique known by a person skilled in the art may be employed. The seed may be coated via a batch or continuous coating process. The seed may be coated with the composition according to the present invention which is either in solid form or liquid form. Preferably, an aqueous dispersion or solution is used.

The seeds may be separated prior to the coating step. In one embodiment, mechanical means, such as a sieve, may be employed for separating the seeds. The separated seeds can then be introduced into a coating machine having a seed reservoir. In one embodiment, the seeds are combined with the composition described herein, optionally with a binder and/or adhesive, in a mixing bowl.

In some aspects, one or more layers of coating which comprises the composition according to the present invention may be added onto the seeds or the agglomeration thereof. Outer layers can be introduced sequentially by coating the seeds or the agglomeration thereof in a rotating drum.

Agglomerators or agglomerator devices may also be utilized. Coating may be performed within a rotary coater by placing the seeds within a rotating chamber, which pushes the seeds against the inside wall of the chamber. Centrifugal forces and mixing bars placed inside the coater allow the seeds to rotate and mix with a coating layer comprising the composition according to the present invention. Binder or other coating materials can be pumped into the proximate center of the coater onto an atomizer disk that rotates along with the coating chamber. Upon hitting the atomizer disk, liquid adhesive is then directed outward in small drops onto the seeds.

Seed coating techniques also include, for example, placing the seeds in a rotating pan or drum. The seeds are then mist with water or other liquid, and then gradually a fine inert powder, e.g., diatomaceous earth, is added to the coating pan. Each misted seed becomes the center of a mass of powder, layers, or coatings that gradually increases in size. The mass is then rounded and smoothed by the tumbling action in the pan, similar to pebbles on the beach. The coating layers are compacted by compression from the weight of material in the pan. Binders often are incorporated near the end of the coating process to harden the outer layer of the mass. Binders can also reduce the amount of dust produced by the finished product in handling, shipping and sowing. Screening techniques, such as frequent hand screening, are often times utilized to eliminate blanks or doubles, and to ensure uniform size. For example, tolerance for seed coating compositions described herein can be +/− 1/64 inch (0.4 mm), which is the US seed trade standard for sizing, established long before coatings were introduced. For example, coated lettuce seed is sown most frequently with a belt planter through an 8/64 inch (3.2 mm) diameter round holes in the belt. This hole size requires that the lettuce seeds coated with the composition according to the present invention can be sized over a 7.5/64 inch (3.0 mm) screen and through an 8.5/64 inch (3.4 mm) screen.

In one embodiment of the present invention, the seed may be contacted with the composition by using an “in situ coating” process, notably by implanting in a hole or a furrow in the soil a seed of a plant, and then applying the composition according to the present invention to surround or partially surround, or to be adjacent to the seed, so that the seed come into contact with the composition, notably with the guar derivative. According to the invention, the hole may notably be a hole, a cavity or a hollowed area. The seed may be one that has not be treated by any agent, or a seed that has been treated with an agrochemical (such as fungicide and insecticide) and that has not been treated with the composition of the present invention. Preferably, the composition is deposited on the carrier to provide a granule or a micro granule before being applied. The granule or the micro granule containing the guar derivative may be prepared by using the methods described above.

In still another embodiment, the guar derivative according to the present invention (or the composition containing said guar derivative) is administered to a soil in which a plant is cultivated. Then the seeds of the plant can be applied to the soil so that the seeds will come into contact with the composition, notably with the guar derivative. Notably, the composition in liquid form, such as in the form of aqueous solution/dispersion, or the composition in solid form, such as in powder or granule, may be used.

Preferably, the application of the seed and the application of the composition according to the present invention are performed mechanically. It is appreciated that either or both of the referenced applications can be performed manually as well.

According to a preferred embodiment, the guar derivative as defined above is used in a liquid form.

In one embodiment of the present invention, the guar derivative is used in an amount ranging from 50 to 500 g/quintal seed.

### Biofungicide

The term “bio fungicide” as used herein means a component controlling or eliminating the fungal activity by biological means, such as by using a microorganism such as bacterium, as opposed to the use of a synthetic chemical agent.

By “microorganism” is meant herein a microscopic organism, which may exist in its single-cell form or as colony of cells. In a particular embodiment, said microorganism is unicellular.

The present invention relates more particularly to soil microorganisms, also known as soil microbes.

According to an embodiment, the microorganisms are fungi, in particular unicellular fungi, or bacteria. In a particular embodiment, the microorganisms are bacteria.

According to an embodiment, the biofungicide of the invention is a suppressive microorganism, that is to say a microorganism having a suppressive action on pathogenic fungi.

“Suppressive microorganism” as used herein means any microorganism that can kill or inhibit the growth of fungi by any means. Suppressive microorganism, for instance suppressive bacteria, can be selected that inhibit or kill pathogenic fungi in numerous ways including changing the pH of the microenvironment to one that inhibits or kills the fungi, and producing harmful byproducts like hydrogen cyanide, antifungal substances, cell-wall degrading (lytic) enzymes, and iron chelating compounds called siderophores.

According to an embodiment, the bacteria according to the invention are chosen from Gram-positive bacteria.

As used herein, the term “gram-positive bacteria” refers to bacterial cells which stain violet (positive) in the Gram stain assay. The Gram stain binds peptidoglycan which is abundant in the cell wall of gram-positive bacteria. In contrast, the cell wall of “gram-negative bacteria” has a thin layer of peptidoglycan, thus gram-negative bacteria do not retain the stain and allow to uptake the counterstain in the Gram stain assay.

Gram-positive bacteria are well-known from the skilled person and include bacteria from the Actinobaculum, Actinomyces, Arthrobacter, Bifidobacterium, Frankia, Gardnerella, Lysinibacillus, Microbacterium, Micrococcus, Micromonospora, Mycobacterium, Nocardia, Rhodococcus, Streptomyces, Bacillus, Clostridium, Listeria, Enterococcus, Lactobacillus, Leuconostoc, Mycoplasma, Ureaplasma, Lactococcus, Paenibacillus, Pediococcus, Acetobacterium, Eubacterium, Heliobacterium, Heliospirillum and Sporomusa genera.

In a particular embodiment, the Gram-positive bacteria are selected from the group consisting in Streptomyces and Bacillus, genera bacteria.

In a particular embodiment, the Gram-positive bacteria are bacteria from the Bacillus genera, in particular bacteria selected from the group consisting of Bacillus pumilus (such as B. pumilus strain GB34 (YieldShield; Bayer), B. pumilus strain QST2808 (Sonata; Bayer) and B. pumilus strain BU F-33), Bacillus firmus (such as B. firmus strain 1-1582 (Votivo and Nortica; Bayer)), Bacillus subtilis (such as B. subtilis strains GB03 (Kodiak; Bayer), MBI 600 (Subtilex; Becker Underwood) and QST 713 (Serenade; Bayer), B. subtilis strain GB122 plus, B. subtilis strain EB120, B. subtilis strain J-P13, B. subtilis FB17, B. subtilis strains QST30002 and QST3004 (NRRL B-50421 and NRRLB -50455), B. subtilis strains QST30002 and QST3004 (NRRL B-50421 and NRRLB-50455) sandpaper mutants, B. subtilis strain QST 713, B. subtilis strain DSM 17231, B. subtilis strain KAS-001, B. subtilis strain KAS-006, B. subtilis strain KAS-009, B. subtilis strain KAS-010, B. subtilis strain KAS-011 and B. subtilis strain CCT0089), Bacillus thuringiensis (such as B. thuringiensis galleriae strain SDS-502, B. thuringiensis kurstaki VBTS 2546, B. thuringiensis subsp. kurstaki strain SA 11, B. thuringiensis subsp. kurstaki strain SA 12, B. thuringiensis subsp. kurstaki strain ABTS 351, B. thuringiensis subsp. kurstaki strain EG 2348, B. thuringiensis subsp. kurstaki strain VBTS 2477 quadruple enterotoxin-deficient mutants, B. thuringiensis subsp. aizawai strain GC 91, and B. thuringiensis subsp. tenebrionis), or bacteria from the Streptomyces genera, in particular bacteria from the Streptomyces K61 species.

In a more particular embodiment, the Gram-positive bacteria are bacteria from the B. subtilis, the B. thuringiensis or the B. megaterium species. In still a particular embodiment, the Gram-positive bacteria are B. subtilis CCT 0089, B. thuringiensis CCT 2335 or B. megaterium CCT 0536.

According to an embodiment, the bacteria according to the invention are chosen from Gram-negative bacteria.

Gram-negative bacteria are well-known from the skilled person and include bacteria from the Acetobacter, Achromobacter, Actinobacillus, Agrobacterium, Allorhizobium, Azospirillum, Azotobacter, Bordetella, Bradyrhizobium, Brucella, Burkholderia, Campylobacter, Carbophilus, Chelatobacter, Chryseobacterium, Citrobacter, Delftia, Enterobacter, Erwinia, Escherichia, Flavobacterium, Francisella, Frateuria, Gluconobacter, Helicobacter, Haemophilus, Kalstia, Klebsiella, Legionella, Mesorhizobium, Moraxella, Neisseria, Pantoea, Pasteurella, Phyllobacterium, Proteus, Pseudomonas, Rhizobium, Salmonella, Serratia, Shigella, Sinorhizobium, Treponema, Vibrio, Xanthomonas and Yersinia genera.

In a particular embodiment, the Gram-negative bacteria are selected from the group consisting in Pseudomonas genera bacteria.

In a particular embodiment, the Gram-negative bacteria are bacteria from the Acetobacter genera, in particular bacteria from the Pseudomonas genera, in particular bacteria selected from the group consisting in Pseudomonas putida, Pseudomonas fluorescens, Pseudomonas protegens, Pseudomonas chlororaphis (such as Pseudomonas chlororaphis strain MA342), Pseudomonas aurantiaca, Pseudomonas mendocina and Pseudomonas rathonis species.

In more particular embodiments, the Gram-negative bacteria are bacteria from the B. japonicum or the P. putida species. In still a particular embodiment, the Gram-negative bacteria are B. japonicum strain CCT 4065 or P. putida CCT 5357.

According to anyone of the invention embodiments, the microorganism may be for instance bacteria chosen from the B. subtilis, the B. megaterium, the B. thuringiensis, the B. japonicum or the P. putida species.

According to an embodiment, the microorganims according to the invention are fungi, in particular unicellular fungi.

Fungi are well-known from the skilled person and include Ascomycetes, Glomeromycetes and Basidiomycetes. In a particular embodiment, said fungi are selected from the Ascomycetes phylum, in particular from the group consisting in the Trichoderma, Metarhizium, Beauveria, Lecanicillium, Purpureocillium, Gliocladium, Isaria, Fusarium, Arthrobotrys, Penicillium, Aspergillus, Ampelomyces, Coniothyrium, Aureobasidium and Candida genera; from the Glomeromycetes phylum, in particular from the group consisting in the Glomus and Rhizophagus genera; and/or from the Basidiomycetes phylum, in particular from the group consisting in the Phlebiopsis and Rhizoctonia genera.

In a particular embodiment, said fungi are fungi from the Trichoderma genera, in particular fungi selected from the group consisting in the Trichoderma viride, Trichoderma atroviride (such as Trichoderma atroviride strain I-1237, Trichoderma atroviride strain SC1, Trichoderma atroviride strain Trichoderma harzianum (such as, Trichoderma harzianum Rifai strain T-22 and ITEM-908), Trichoderma asperellum (such as Trichoderma asperellum strain ICC012 T25 and TV1, Trichoderma asperellum strain T34); fungi from the Metarhizium genera, in particular fungi selected from the group consisting in the Metarhizium anisopliae, such as Metarhizium anisopliae var. anisopliae BIPESCO 5/F52; fungi from the Beauveria genera, in particular fungi from the Beauveria bassiana species (such as Beauveria bassiana strain ATCC 74040, Beauveria bassiana strain GHA, Beauveria bassiana strain NPP111B005, and Beauveria bassiana strain 147); fungi from the Lecanicillium genera, in particular fungi selected from Lecanicillium muscarium species, such as Lecanicillium muscarium strain Ve6; fungi from the Gliocladium genera, in particular fungi from the Gliocladium catenulatum species, such as Gliocladium catenulatum strain J1446; fungi from the Isaria genera, in particular fungi from the Isaria fumosorosea species, such as Isaria fumosorosea Apopka strain 97; fungi from the Ampelomyces genera, in particular fungi from the Ampelomyces quisqualis species; fungi from the Candida genera, in particular fungi from the Candida oleophila species; fungi from the Phlebiopsis genera, in particular fungi from the Phlebiopsis gigantea species.

In more particular embodiments, the fungi are fungi from the Trichoderma harzianum species. In still a particular embodiment, the fungi are Trichoderma harzianum CCT 4790.

According to anyone of the invention embodiments, the microorganism may be for instance bacteria chosen from the B. subtilis, the B. megaterium, the B. thuringiensis, the B. japonicum or the P. putida species or fungi from the T. harzianum species, such as those described previously.

The amount of microorganism to be used may vary from one microorganism to another and may also depend on the seed to be treated. In one embodiment of the present invention, the microorganism is used in an amount ranging from 1.10⁴ to 1.10¹⁵ CFU/quintal seed.

The present invention also relates to a method for maintaining or increasing the growth rate and/or biofungicidal activity of such microorganisms, in particular of suppressive microorganism, comprising a step of contacting at least one seed with a guar derivative as defined above.

According to a preferred embodiment, this method is carried out in liquid medium. Therefore, preferably, this method comprises a step of contacting at least one seed with a guar derivative as defined above in a liquid form or with a liquid composition comprising a guar derivative as defined above.

The present invention also relates to the use of a microorganism, in particular a suppressive microorganism, and of a guar derivative as defined above, as bio fungicide.

Therefore, the present invention relates to the combined use of said microorganism, in particular suppressive microorganism, and guar derivative. It has been shown that the combination of said microorganism, in particular suppressive microorganism, and guar derivative gives a bio fungicide activity.

The present invention also relates to a biofungicide composition comprising at least one microorganism, in particular a suppressive microorganism, and at least one guar derivative as defined above.

According to anyone of the invention embodiments, the microorganism and the guar derivative are combined in a ratio microorganism:guar derivative ranging from 1.10⁴ to 1.10¹⁵, for example ranging from 1.10⁴ to 1.10¹², for example ranging from 1.10⁴ to 1.10¹¹ CFU/g, for example ranging from 1.10⁴ to 5.10¹⁰ CFU/g, for example ranging from 1.10⁵ to 1.10¹⁰ CFU/g. For instance, the microorganism and the guar derivative may be combined in a ratio microorganism: guar derivative ranging from 1.10⁸ to 1.10¹².

Preferably, this bio fungicide composition is in a liquid form.

The present invention also relates to a kit comprising at least one microorganism, in particular a suppressive microorganism, and at least one guar derivative as defined above, said kit being preferably used as biofungicide.

The present invention thus also relates to the use of the above-mentioned kit as bio fungicide.

The present invention also relates to a seed coated with the biofungicide composition as defined above.

In one embodiment, the seed is of the crop or plant species including but not limited to corn (Zea mays), Brassica sp. (e.g., B. napus, B. rapa, B. juncea), alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus animus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, woody plants such as conifers and deciduous trees, squash, pumpkin, hemp, zucchini, apple, pear, quince, melon, plum, cherry, peach, nectarine, apricot, strawberry, grape, raspberry, blackberry, soybean, sorghum, sugarcane, rapeseed, clover, carrot, and Arabidopsis thaliana.

In one embodiment, the seed is of any vegetables species including but not limited to tomatoes (Lycopersicon esculentum), lettuce (e.g., Lactuca sativa), green beans (Phaseolus vulgaris), lima beans (Phaseolus limensis), peas (Lathyrus spp.), cauliflower, broccoli, turnip, radish, spinach, asparagus, onion, garlic, pepper, celery, and members of the genus Cucumis such as cucumber (C. sativus), cantaloupe (C. cantalupensis), and musk melon (C. melo).

In one embodiment, the seed is of any ornamentals species including but not limited to hydrangea (Macrophylla hydrangea), hibiscus (Hibiscus rosasanensis), petunias (Petunia hybrida), roses (Rosa spp.), azalea (Rhododendron spp.), tulips (Tulipa spp.), daffodils (Narcissus spp.), carnation (Dianthus caryophyllus), poinsettia (Euphorbia pulchenima), and chrysanthemum.

In one embodiment, the seed is of any conifer species including but not limited to conifers pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

In one embodiment, the seed is of any leguminous plant species including but not limited beans and peas. Beans include guar, locust bean, fenugreek, soybean, garden beans, cowpea, mungbean, lima bean, fava bean, lentils, chickpea, pea, moth bean, broad bean, kidney bean, lentil, dry bean, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Typical forage and turf grass for use in the methods described herein include but are not limited to alfalfa, orchard grass, tall fescue, perennial ryegrass, creeping bent grass, lucerne, birdsfoot trefoil, clover, stylosanthes species, lotononis bainessii, sainfoin and redtop. Other grass species include barley, wheat, oat, rye, orchard grass, guinea grass, sorghum or turf grass plant.

In another embodiment, the seed is selected from the following crops or vegetables: corn, wheat, sorghum, soybean, tomato, cauliflower, radish, cabbage, canola, lettuce, rye grass, grass, rice, cotton, sunflower and the like. In another embodiment, the seed is selected from corn, wheat, barley, rice, peas, oats, soybean, sunflower, alfalfa, sorghum, rapeseed, sugar beet, cotton, tobacco, forage crops, linseed, hemp, grass, vegetables, fruits and flowers seeds.

It is understood that the term “seed” or “seedling” is not limited to a specific or particular type of species or seed. The term “seed” or “seedling” can refer to seed from a single plant species, a mixture of seed from multiple plant species, or a seed blend from various strains within a plant species. In one embodiment, crop seeds include but are not limited to rice, corn, wheat, barley, oats, soybean, cotton, sunflower, alfalfa, sorghum, rapeseed, sugarbeet, tomato, bean, carrot, tobacco or flower seeds.

An aspect of the invention includes methods of using the bio fungicide composition of the invention to control, prevent or reduce pathogenic fungus infestations in growing plants, on seeds, and on harvested crops.A biofungicide composition of the invention can be used to treat plants, seeds, plants, leaves, cuttings, and plant media and for post-harvest treatment of crops.

As mentioned previously, the present invention also relates to a bio fungicide composition comprising at least one microorganism, in particular a suppressive microorganism, and at least one guar derivative as defined above.

According to a preferred embodiment, the composition is applied onto the foliar system of the plant. Such application is preferably carried out by spraying a composition as disclosed above onto the leaves of the plant. For example, the composition can be sprayed onto a field using appropriate means well known in agriculture.

For instance, a biofungicide composition of the present invention may be dilute enough to be readily sprayed using standard agricultural spray equipment. Application of the composition to foliage may be accomplished by spraying, using any conventional means for spraying liquids, such as spray nozzles, atomizers or the like. The composition of the invention can be used in precision farming techniques, in which apparatus is employed to vary the amount of pesticide applied to different parts of a field, depending on variables such as the particular plant species present, soil composition, etc. In one embodiment of such techniques, a global positioning system operated with the spraying apparatus can be used to apply the desired amount of the composition to different parts of a field. The selection of application rates that are effective for a composition of the invention is within the skill of the ordinary agricultural scientist.

According to a preferred embodiment, the present invention relates to a method for treating a plant wherein a composition as defined previously is applied onto at least one part of said plant.

The composition may be applied directly onto the plant, or may be diluted just before application with a liquid diluent comprising water or a mixture of water and organic solvent, or may be mixed just before application with another agrochemical composition.

In one embodiment, the composition may be applied onto the foliar system of the plant, preferably by spraying said composition onto the leaves of the plant.

The present invention is not limited to bio fungicide compositions but also relates to any microorganism useful in agricultural applications, such as biopesticides and the like.

The following examples are included to illustrate embodiments of the invention, but is not limited to described examples.

### EXAMPLES

### Example 1:

The following materials are used in the experiments:

Guar: a guar hydroxypropyltrimonium chloride having an average molecular weight between 5,000 and 25,000 Daltons, a DS of 0.2, and a MS between 0.2 and 1.0, available from Solvay (provided as a powder)

Bacteria strains were acquired from Tropical Culture Collection in André Tosello Foundation—Brazil.
Bacillus subtilis CCT 0089
Bacillus megaterium CCT 0536
Bradyrhizobium japonicum CCT 4065

All strains were stored at −80° C. in the appropriated culture media, containing 15% of glycerol.

Two different culture media were used in the experiments:
NA media containing per liter: 3 g of meat extract, 5 g of peptone and 15 g of agar (for solid media only)
YMA media containing per liter: 0.5 g of monobasic potassium phosphate, 0.2 g of magnesium sulphate; 0.1 g of sodium chloride; 0.5 g of yeast extract; 10 g of mannitol (for inoculum and solid media only); 5 mL of a 5% bromothymol blue solution and 15 g of agar (solid media only).
   For strains Bacillus subtilis and Bacillus megaterium, NA media was used. For strain Bradyrhyzobium japonicum, YMA media was used. These media were selected according to strains supplier.

A 250 mL shake flask containing 100 mL of NA or YMA culture media, was inoculated with 1 mL of the stock culture and incubated at 30° C., 150 rpm for 72 hours.

For each strain, 10 mL of the reactivation media were then transferred into a 250 mL shake flask containing 100 mL of the same media, with the addition of guar powder (at 1 wt % in the incubation media); and incubated at 30° C., 150 rpm, for 96 hours. An experiment without addition of guar powder is also performed for each strain as a control.

100 μL samples of each experiments were taken after 0 h, 24 h, 48 h, 72 h and 96 h of incubation. These samples were diluted (the dilutions were variable according to strain growth, being from 1×10⁻⁵ up to 1×10⁻¹⁵) and the dilutions plated in solid NA or YMA media. The plates were incubated at 30° C. until appearance of colonies. After incubation, the number of colonies present in each dilution was counted and used to evaluate bacterial growth.

For bacterial growth rate determination, a graph of the log₁₀ (number of colonies) versus time of incubation was constructed. The straight line in this graph represents the exponential phase of bacterial growth and the angular coefficient represents the bacterial growth rate (μ).

The μ value was used to compare all the experiments and to evaluate the influence of guar addition on bacterial growth.

For this set of experiments the ratio of microorganisms and guar is equal to 3.50×10⁴ CFU/g. The bacteria growth rate (μ) obtained for the different experiments are summarized in Table 1:

TABLE 1

| Composition | Bacteria growth rate (h⁻¹) |
|---|---|
| Bacillus subtilis CCT 0089 | 0.0647 |
| Bacillus subtilis CCT 0089 + guar | 0.0739 |
| Bacillus megaterium CCT 0536 | 0.0605 |
| Bacillus megaterium CCT 0536 + guar | 0.0690 |
| Bradyrhyzobium japonicum CCT 4065 | 0.0891 |
| Bradyrhyzobium japonicum | 0.0880 |
| CCT 4065 + guar | |

For the three strains, a comparable or higher value of bacteria growth rate is obtained in presence of guar. The addition of guar permits to maintain or increase the growth rate of these different strains of bacteria. In Table 2 are reported the relative increase or decrease of bacteria growth rate with the addition of guar compared to the control for each strain. An increase of bacteria growth rate of +14% is observed for the two gram positive bacteria (Bacillus subtilis and Bacillus megaterium), whereas a nil value is observed for Bradyrhyzobium japonicum, which correspond to a comparable growth rate of bacteria with and without guar.

TABLE 2

| | Relative increase of bacteria |
|---|---|
| Strain | growth rate with guar addition |
| Bacillus subtilis CCT 0089 | 14% |
| Bacillus megaterium CCT 0536 | 14% |
| Bradyrhyzobium japonicum CCT 4065 | 0% |

A similar experiment was conducted at a ratio bacteria/guar equals to 7.00×10⁵ on the same bacteria species, a comparable or higher value of bacteria growth is obtained in presence of guar.

### Example 2:

The following materials are used in the experiments:

Guar: a guar hydroxypropyltrimonium chloride having an average molecular weight between 5,000 and 25,000 Daltons, a DS of 0.2, and a MS between 0.2 and 1.0, available from Solvay (provided as a powder)

Liquid guar formulation: an aqueous formulation of the powder guar at 25% from Solvay

Bacteria strains were acquired from Tropical Culture Collection in André Tosello Foundation—Brazil.
Bacillus subtilis CCT 0089
Bacillus megaterium CCT 0536
Bradyrhizobium japonicum CCT 4065

All strains were stored at −80° C. in the appropriated culture media, containing 15% of glycerol.

Two different culture media were used in the experiments:
NA media containing per liter: 3 g of meat extract, 5 g of peptone and 15 g of agar (for solid media only)
YMA media containing per liter: 0.5 g of monobasic potassium phosphate, 0.2 g of magnesium sulphate; 0.1 g of sodium chloride; 0.5 g of yeast extract; 10 g of mannitol (for inoculum and solid media only); 5 mL of a 5% bromothymol blue solution and 15 g of agar (solid media only).

For strains Bacillus subtilis and Bacillus megaterium, NA media was used. For strain Bradyrhyzobium japonicum, YMA media was used. These media were selected according to strains supplier.

A 250 mL shake flask containing 100 mL of NA or YMA culture media, was inoculated with lmL of the stock culture and incubated at 30° C., 150 rpm for 72 hours.

For each strain, 10 mL of the reactivation media were then transferred into a 250 mL shake flask containing 100 mL of the same media, with the addition of guar powder or guar liquid formulation; and incubated at 30° C., 150 rpm, for 96 hours. An experiment without addition of guar powder is also performed for each strain as a control.

100 μL samples of each experiments were taken after 0 h, 24 h, 48 h, 72 h and 96 h of incubation. These samples were diluted (the dilutions were variable according to strain growth, being from 1×10⁻⁵ up to 1×10⁻¹⁵) and the dilutions plated in solid NA or YMA media. The plates were incubated at 30° C. until appearance of colonies. After incubation, the number of colonies present in each dilution was counted and used to evaluate bacterial growth.

For bacterial growth rate determination, a graph of the log₁₀(number of colonies) versus time of incubation was constructed. The straight line in this graph represents the exponential phase of bacterial growth and the angular coefficient represents the bacterial growth rate (μ).

The μ value was used to compare all the experiments and to evaluate the influence of the two guars addition on bacterial growth. For this set of experiments the ratio of microorganisms and guar was set at 1.0×10¹⁰ CFU/g. The bacteria growth rate (μ) obtained for the different experiments are summarized in Table 3:

TABLE 3

| | Bacteria growth |
|---|---|
| Composition | rate (h⁻¹) |
| Bacillus subtilis CCT 0089 | 0.0862 |
| Bacillus subtilis CCT 0089 + guar powder | 0.0931 |
| Bacillus subtilis CCT 0089 + guar liquid | 0.0975 |
| formulation | |
| Bacillus megaterium CCT 0536 | 0.0834 |
| Bacillus megaterium CCT 0536 + guar | 0.1018 |
| powder | |
| Bacillus megaterium CCT 0536 + guar liquid | 0.0958 |
| formulation | |
| Bradyrhyzobium japonicum CCT 4065 | 0.0915 |
| Bradyrhyzobium japonicum CCT 4065 + | 0.0936 |
| guar powder | |
| Bradyrhyzobium japonicum CCT 4065 + | 0.0913 |
| guar liquid formulation | |

For the three strains, a comparable or higher value of bacteria growth rate is obtained in presence of the guar under powder form and the guar in aqueous formulation. The addition of guar permits to maintain or increase the growth rate of these different strains of bacteria. In Table 4 are reported the relative increase or decrease of bacteria growth rate with the addition of the two guar grades compared to the control for each strain. An increase of bacteria growth rate ranging from 8% to 22% is observed for the two gram positive bacteria (Bacillus subtilis and Bacillus megaterium), whereas a nil value or 2% is observed for Bradyrhyzobium japonicum, which correspond to a comparable growth rate of bacteria with and without guar.

TABLE 4

| | Relative increase |
|---|---|
| | of bacteria |
| | growth rate |
| Strain | with guar addition |
| Bacillus subtilis CCT 0089 + guar | 8% |
| powder | |
| Bacillus subtilis CCT 0089 + guar liquid | 13% |
| formulation | |
| Bacillus megaterium CCT 0536 + guar | 22% |
| powder | |
| Bacillus megaterium CCT 0536 + guar | 15% |
| liquid formulation | |
| Bradyrhyzobium japonicum CCT 4065 + | 2% |
| guar powder | |
| Bradyrhyzobium japonicum CCT 4065 + | 0% |
| guar liquid formulation | |

### Example 3:

The following materials are used in the experiments:

Guar: a guar hydroxypropyltrimonium chloride having an average molecular weight between 5,000 and 25,000 Daltons, a DS of 0.2, and a MS between 0.2 and 1.0, available from Solvay (provided as a powder)

Bacteria strains were acquired from Tropical Culture Collection in André Tosello Foundation—Brazil:
Bacillus thuringiensis CCT 2335
Pseudomonas putida CCT 2357

All strains were stored at −80° C. in the appropriate culture media, containing 15% of glycerol.

Only one culture media was used for both strains
NA media containing per liter: 3 g of meat extract, 5 g of peptone and 15 g of agar (for solid media only)

A 250 mL shake flask containing 100 mL of NA or YMA culture media, was inoculated with 1 mL of the stock culture and incubated at 30° C., 150 rpm for 72 hours.

For each strain, 10 mL of the reactivation media were then transferred into a 250 mL shake flask containing 100 mL of the same media, with the addition of guar powder or guar liquid formulation; and incubated at 30° C., 150 rpm, for 96 hours. An experiment without addition of guar powder is also performed for each strain as a control.

100 μL samples of each experiments were taken after 0 h, 24 h, 48 h, 72 h and 96 h of incubation. These samples were diluted (the dilutions were variable according to strain growth, being from 1×10⁻⁵ up to 1×10⁻¹⁵) and the dilutions plated in solid NA media. The plates were incubated at 30° C. until appearance of colonies. After incubation, the number of colonies present in each dilution was counted and used to evaluate bacterial growth.

For bacterial growth rate determination, a graph of the logᵢₒ(number of colonies) versus time of incubation was constructed. The straight line in this graph represents the exponential phase of bacterial growth and the angular coefficient represents the bacterial growth rate (μ).

The μ value was used to compare all the experiments and to evaluate the influence of the two guars addition on bacterial growth. For this set of experiments the ratio of microorganisms and guar was set at 1.0×10⁵ CFU/g. The bacteria growth rate (μ) obtained for the different experiments are summarized in Table 5:

TABLE 5

| | Bacteria growth |
|---|---|
| Composition | rate (h⁻¹) |
| Bacillus thuringiensis CCT 2335 | 0.0898 |
| Bacillus thuringiensis CCT 2335 + guar | 0.1028 |
| Pseudomonas putida CCT 5357 | 0.1133 |
| Pseudomonas putida CCT 5357 + guar | 0.1282 |

For the two strains, a higher value of bacteria growth rate is obtained in the presence of guar than without guar addition. The addition of guar permits to increase the growth rate of these different strains of bacteria. In Table 6 are reported the relative increase of bacteria growth rate with the addition of guar compared to the control for each strain. An increase of bacteria growth rate ranging from 13% to 14% is observed for the two strains of bacteria.

TABLE 6

| | Relative increase of |
|---|---|
| | bacteria growth rate |
| Strain | with guar addition |
| Bacillus thuringiensis CCT 2335 | 14% |
| Pseudomonas putida CCT 5357 | 13% |

### Example 4:

The following materials are used in the experiments:

Guar: a guar hydroxypropyltrimonium chloride having an average molecular weight between 5,000 and 25,000 Daltons, a DS of 0.2, and a MS between 0.2 and 1.0, available from Solvay (provided as a powder)

Liquid guar formulation: an aqueous formulation of the powder guar at 25% from Solvay

All microorganisms strains were acquired from Tropical Culture Collection in André Tosello Foundation—Brazil, some of them have reference in American Type Culture Colection (ATCC).
Trichoderma harzianum CCT 4790

All strains were stored at −80° C. in the appropriate culture media, containing 20% of glycerol.

Culture media used in the experiments:
Nutrient broth (NA) media containing per liter: 3 g of meat extract, 5 g of peptone and 15 g of agar (for solid media only)
Oatmeal Agar (OA) containing per liter: 25 g of oat flakes or flour and 15 g of agar

The media OA was used for reactivation of the strain T. harzianum according to supplier's recommendation.

For the experiments with guar, only NA media was used.

Reactivation of Microorganisms:

A Petri dish containing 20 mL of OA media was used for the reactivation of the strain T. harzianum.

The stock culture was used to inoculate the solid media for the strain T. harzianum and the petri dishes were incubated at 25° C. until complete growth.

Incubation with Guar:

From the reactivation media on petri dish, the spores of fungi were recovered and a spore solution was prepared.

500 μL of the spore solution (approximately 1×10¹⁰ CFU/mL) were transferred to Erlenmeyer flasks containing 50 mL of media (controls and NA media with guar) and incubated at 25° C. Samples were taken at 48 h, 120 h and 168 h, filtered on filter paper and incubated at 60° C. before weighing

*Control media=NA without guar addition

Growth Evaluation:

The dry biomass recovery after each sample was plotted in a graphic dry biomass vs time and the growth curve could be obtained.

The growth rate (μ) was calculated considering only the exponential phase of the growth and compared with the control.

The μ value was used to compare all the experiments and to evaluate the influence of guar addition on fungi growth. The microorganisms growth rate (μ) obtained for the different experiments are summarized in Table 7:

TABLE 7

| | Fungi growth |
|---|---|
| Composition | rate (h⁻¹) |
| Trichoderma harzianum CCT 4790 | 0.0009 |
| Trichoderma harzianum CCT 4790 + | 0.0013 |
| guar powder | |
| Trichoderma harzianum CCT 4790 + | 0.0015 |
| guar liquid formulation | |

A higher value of growth rate is obtained in presence of the guar under powder form and the guar in aqueous formulation compared to control. Hence, the addition of guar permits to increase the growth rate of this strain of fungi. In Table 8 are reported the relative increase of growth rate with the addition of the two guar grades compared to the control. An increase of bacteria growth rate ranging from 44% to 67% is observed for this strain of fungi.

TABLE 8

| | Relative increase of |
|---|---|
| | fungi growth rate |
| Strain | with guar addition |
| Trichoderma harzianum CCT 4790 + | 44% |
| guar powder | |
| Trichoderma harzianum CCT 4790 + | 67% |
| guar liquid formulation | |

1. A fungicide composition comprising a bio-fungicide and a guar derivative containing at least one hydroxyalkyl group.

2. The fungicide composition of claim 1, wherein the bio-fungicide is a microorganism.

3. The fungicide composition of claim 1, wherein the guar derivative further contains at least one cationic group.

4. A method for maintaining or increasing the growth rate of microorganisms, the method comprising a step of contacting said microorganism with a guar derivative containing at least one hydroxyalkyl group.

5. The method of claim 4, wherein the guar derivative further contains at least one cationic group.

6. (canceled)

7. (canceled)

8. A kit comprising at least one microorganism and at least one guar derivative containing at least one hydroxyalkyl group.

9. The kit of claim 8, wherein the guar derivative further contains at least one cationic group.

10. (canceled)

11. A method for controlling fungal organism comprising applying a fungicide composition according to claim 1.

12. A method of controlling or preventing infection of a plant by phytopathogenic fungi, comprising the step of applying to said plant a fungicide composition according to claim 1.

13. A method for treating a plant wherein a composition according to claim 1 is applied onto at least one part of said plant.

14. The method of claim 13, wherein the composition is applied onto the foliar system of the plant.

15. The fungicide composition of claim 2, wherein the microorganism is fungi or bacteria.

16. The method of claim 4, wherein the microorganisms are suppressive microorganisms.

17. The method of claim 14, wherein the composition is applied onto the foliar system of the plant by spraying said composition onto the leaves of the plant.
